(19) United States
(12) Patent Application Publication
(10) Pub. No.: US 2008/0032059 A1
(43) Pub. Date: Feb. 7, 2008

(54) Device and Process for Plasma Coating/Sterilization

(75) Inventors: Johann Zimmerer, Bernhardswald (DE); Heinz Humele, Thalmassing (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: KRONES AG, Neutraubling (DE)

(21) Appl. No.: 11/632,763

(22) PCT Filed: Jul. 16, 2005
(86) PCT No.: PCT/EP05/07773

§ 371 Date: Apr. 2, 2007

(30) Foreign Application Priority Data
Jul. 24, 2004 (DE) 10 2004 036 063.4

(51) Int. Cl. A61L 2/14; B05D 3/06; B65B 55/08; H01H 37/32

(52) U.S. Cl. 427/458; 118/715; 118/719; 118/729; 118/733; 422/186.05; 422/28; 422/310

(57) ABSTRACT

A device for treating containers such as bottles, preferably PET containers, such as PET bottles, with a plasma, whereby the device is designed for sterilizing and/or coating the containers. In addition, the device also relates to a method for treating containers, preferably PET containers such as PET bottles, with a plasma, whereby the treatment comprises sterilization and/or the coating of the containers. Also provided is an airlock for containers such as bottles, in particular PET containers such as PET bottles, having cells to receive the containers, at least one cell being designed to receive at least two containers.

### CROSS-REFERENCE TO RELATED APPLICATION

This is the U.S. national stage under 35 U.S.C. §371, of international application no. PCT/EP2005/007773, having an international filing date of Jul. 16, 2005, and claims priority to German application no. 10 2004 036 063.4 filed on Jul. 24, 2004.

### FIELD OF THE DISCLOSURE

The disclosure relates to a device and a method for treating containers, especially PET containers such as PET bottles, with a plasma.

### BACKGROUND OF THE DISCLOSURE

DE 698 15 359 T2 describes a method in which plastic containers can be provided with a gas barrier layer on the outside. To do so, a coating material is vaporized and the plastic containers are guided in various orientations over the vaporization source.

Disadvantages here include the great mechanical complexity and the inaccurate distribution of the coating on the irregularly shaped bottle bodies.

Such gas barrier layers serve to delay the volatilization of CO₂ from beverages, for example, to thereby increase the minimum shelf-life.

DE 101 34 037 describes a device for plasma sterilization of containers, especially PET bottles in a plasma chamber where PET bottles are fed into a plasma chamber and removed from it and sterilized by means of a plasma. This may take place directly before filling the bottles, for example, to keep the contents free of microorganisms.

EP 1 009 710 B1 also discloses a device for feeding containers into a treatment space.

### SUMMARY OF THE DISCLOSURE

The object of the present disclosure is to improve upon the known devices for treating containers.

An improvement on the state of the art can be achieved, for example, by designing the device or the method so that the containers are both sterilized and coated. The order of the processing operations of sterilizing and coating may be selected as desired. It is also conceivable for the sterilization to take place simultaneously with the coating and/or for the coating parameters to be selected so that sterilization is also performed at the same time. If the processes take place separately from one another, it is nevertheless conceivable for them to be carried out in one and the same treatment chamber. The coating is preferably performed on the inside of the containers.

An embodiment in which the device or the method is designed so that it can be used either for sterilizing or for coating, depending on the process parameters such as process gases, pressure, plasma generating power, etc., is also advantageous.

In an especially advantageous embodiment, only one plasma treatment area is provided, that area taking the form of a ring or a ring segment through which the containers can be guided. This allows a simple design for conveyance of the containers because the containers can then be guided through the plasma treatment area, which is in the form of a ring segment, with the bottles suspended from a rotor, for example, and this increases the efficiency in comparison with a process using individual chambers or double chambers for treatment of the containers.

Since a great many holders or grippers for the containers are required here, it is advantageous to design the grippers so that one gripper can simultaneously grip several containers, e.g., two or three at the same time. It is thus not necessary to provide one gripper which must have various mechanical adjustment mechanisms for each individual container.

An embodiment in which the containers can be varied in height is also preferred. This makes it possible to place the containers in a treatment position. In particular in the case when an electrode, a microwave conductor or a tube for supplying a process gas is to be arranged in the container, the container with the opening situated upward may be shifted upward or downward to such an extent that the electrode, the microwave conductor or the tube is at least partially inside the container. The situation is similar with a horizontal movement of a horizontal bottle or with an arrangement in which the bottle is situated with the opening upward so that it is inverted over the material or the electrode with a downward movement.

An embodiment in which a hollow body is provided in the treatment chamber, moving along with the containers, is especially advantageous. This allows passages from atmospheric pressure to a low pressure in the treatment chamber through a wall rotating with the bottles. The transition from a stationary supply line to a rotary supply line can then be accomplished under normal atmospheric pressure conditions.

In addition, a treatment chamber in which the bottom is adapted to the path of the containers such that the interior of the plasma treatment area is kept as small as possible is also advantageous. This is advantageous in producing a vacuum in the treatment chamber, for example.

In an advantageous embodiment, airlocks are provided for transferring the containers to be treated from atmospheric pressure into a low-pressure treatment space. In the course of the movement of the containers through the airlock, reduced pressure is generated with the help of pumps. Then several pumps may be provided on the circumference of the airlock and a central pump which generates the vacuum by means of connections at the circumference of the airlock may be used.

In addition, airlocks which have specially designed cells are also advantageous. These cells can accommodate several bottles at the same time and thus allow a higher bottle throughput with a simplified design.

If the height of the containers in the treatment chamber must be adjusted to bring them into the treatment position, for example, then it is advantageous for the containers in the airlocks to be guided at a different height level than that in the treatment chamber during the treatment. The containers may be transferred directly from the airlocks to the treatment chamber already at the input height.

### BRIEF DESCRIPTION OF THE DRAWINGS

In addition, UV lamps are also advantageous for treatment of the containers. These UV lamps may be helpful in sterilization, for example.

Embodiments of the device of this method are explained on the basis of the accompanying figures, in which

FIG. 1 shows a schematic top view of a device for treating containers;

FIG. 2 shows a schematic top view of an airlock;

FIG. 3 shows a schematic top view of the treatment chamber;

FIG. 4 shows a three-dimensional schematic view of the device for treating containers;

FIG. 5 shows a schematic sectional view of the device for treating the containers;

FIG. 6 shows a schematic three-dimensional view of a double gripper.

### DETAILED DESCRIPTION

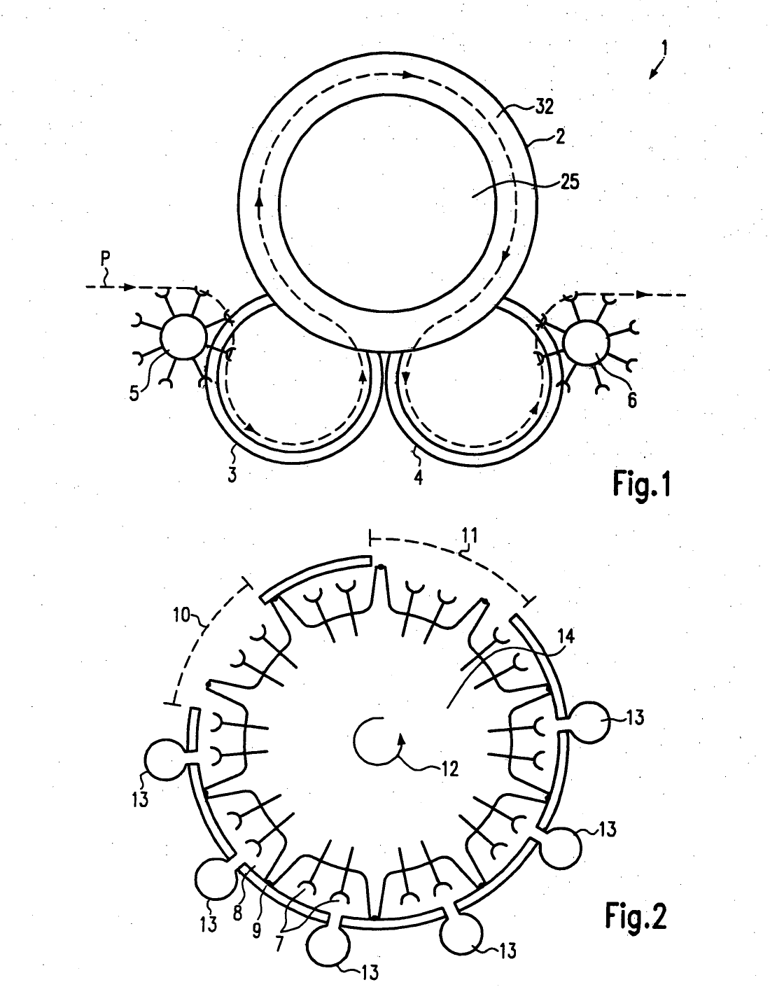

FIG. 1 shows a top view of a device 1 for treating containers. The path provided for the containers is indicated by the dotted line P. From a feed conveyor (not shown), the containers are transferred by a transfer star 5 to an input airlock chamber 3, from which the containers may go to the treatment chamber 2 to be discharged through the airlock 4 after a passage through the treatment chamber and be transferred by transfer star 4 [sic; 6] to a discharge conveyor (not shown).

The treatment chamber 2 has a plasma treatment area 32 in which the containers are able to revolve. A hollow body 25 is arranged in the treatment chamber 2. This yields a ring-shaped plasma treatment area 32. The hollow body 25 advantageously reduces the volume of the plasma treatment area 32 to be evacuated. The greater the size of the hollow body, the smaller the space of the plasma treatment area 2. The airlocks 3 and 4 and the treatment chambers 2 have rotating conveyor means with which the containers can be conveyed on circular segment sections and with which they can be transferred between the airlocks 3, 4 and the treatment chamber 2.

FIG. 2 shows a detailed top view of the airlock 3. The airlock 4 has a similar design in principle except that in it the containers are not evacuated but instead are flooded. In an area 10, shown with dotted lines in FIG. 2, containers can be introduced into the airlock 3. Grippers 7 which are shown schematically are provided to hold the containers. The grippers 7 may be designed so that they are operated from the outside to receive or discharge the bottles. During the revolution between the feed location 10 and the discharge location 11, the grippers are locked so that the containers are securely held.

It is advantageous here that the grippers 7 are designed so that they grip the PET bottles below their collar.

A rotor 14 which can rotate in direction 12 is provided in the airlock 3. This means that the containers are conveyed from the feed area 10 along the longer circumference to the discharge location 11. This yields the longest possible path in the airlock to go from atmospheric pressure at the feed area 10 to a low pressure at the discharge area 11. The central rotor has cells 8 which serve to hold the containers. The various cells 8 are sealed with respect to one another with gaskets 9 vertically and with dynamic gaskets (not shown) horizontally, whereby the gaskets 9 seal the rotor 14 with respect to the outside wall and the dynamic gaskets (not shown) seal the rotor 14 with respect to the bottom and the top. The cells, which are shown in the lower area in FIG. 2, for example, are thus hermetically sealed from the outside. The cells can be pumped out by means of vacuum pumps 13 which are distributed along the outside of the airlock.

The pressure in the cells 8 becomes lower and lower with progressive evacuation on the path from the feed area 10 to the discharge area 11 in the direction of rotation 12. The cells 8 are designed so that each can accommodate two containers. Accordingly, two grippers 7 are provided per cell 8. This allows tighter stacking of the containers in the airlock 3. Therefore, with a uniform rotational speed, a higher throughput is achieved with a lower design expense for the cells.

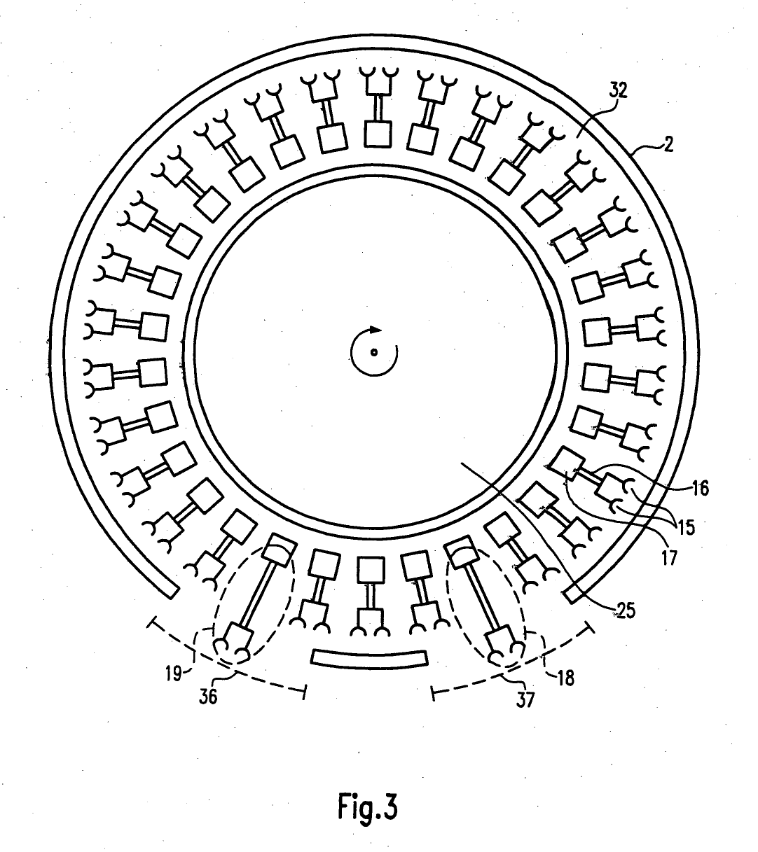

FIG. 3 shows a top view of the treatment chamber 2. The treatment chamber 2 has a plurality of double grippers. Each double gripper has two grip elements 15, each of which can grip a container. The grip elements 15 are arranged on a holding rod 16 which is in turn mounted on a guide 17. The double grippers are arranged along a rotor which can revolve in the treatment chamber 2.

In an area 36 the containers can be transferred to the grippers of the treatment chamber 2. To do so, a double gripper is moved out (as represented by reference numeral 19) so that it reaches the airlock chamber shown in FIG. 2, so that it can receive containers arriving there. Accordingly, the containers can be moved out radially with the double gripper after a revolution in the treatment chamber 2 (as indicated by reference numeral 18) so that they can be picked up by the grippers of an output airlock. The central hollow body 25 is arranged in the space within the double grippers arranged in the form of a ring. This hollow body may be the rotor to which the double grippers are attached.

The grippers 15 are arranged so that the distance between the containers being held and the neighboring containers is always the same. Therefore a uniform treatment of all containers is ensured.

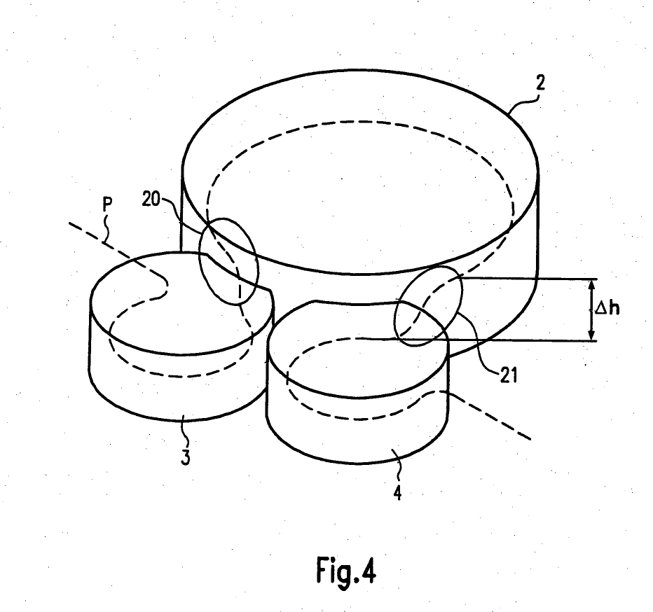

FIG. 4 shows a schematic diagram of the device for treating the containers in a three-dimensional view. The path P of the containers is indicated with a dotted line in FIG. 1.

As shown in FIG. 4, the airlocks 3, 4 are situated at a lower level than the treatment chamber 2. Therefore the containers can be introduced into the treatment chamber 2 at a relatively low level and raised in area 20 there. In area 21 the containers can be lowered onto their path so that they can be discharged to the airlock 4. The containers inside the treatment chamber 20 can be brought into their treatment position by raising the containers in area 20. Likewise they can be removed from the treatment position in area 21 so that the transfer to the airlock chamber 4 can take place.

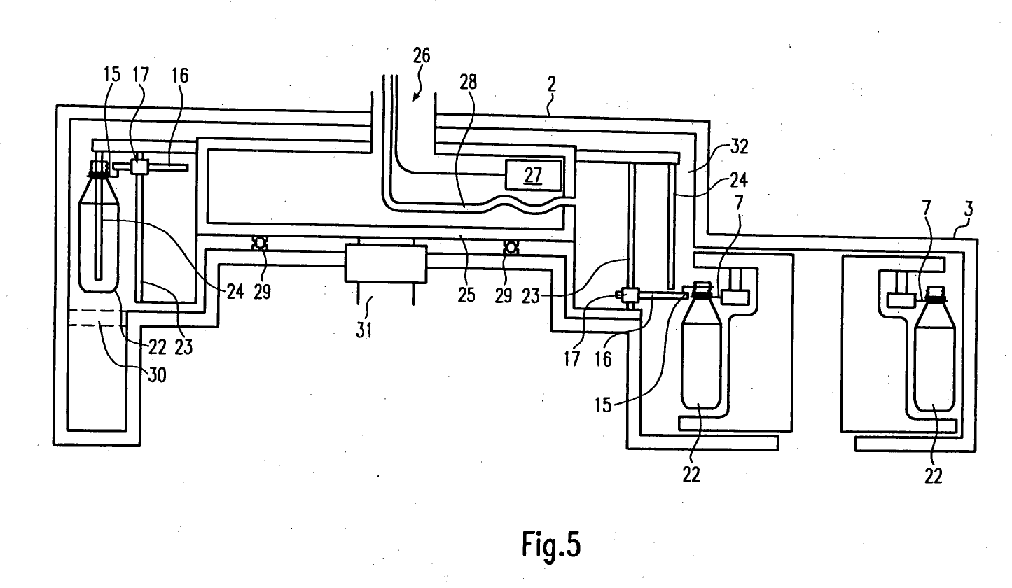

FIG. 5 shows a schematic sectional diagram of-the treatment chamber 2 and the airlock chamber 3.

A gripper 7 which holds a container 22 is provided in the airlock chamber 3. As this shows, the container 22 is held below a carrying edge. This makes it possible for the gripper 15 of the treatment chamber 2 to grip the container above the carrying edge so that the entire container is freely accessible below the carrying edge, which can be advantageous for coating.

As shown in FIG. 5, the grippers 15 are attached to a non-twisting rod 16, illustrated in FIG. 5 in an extracted position so that the gripper 15 can grip the container 22 in the airlock chamber 3. The rod 16 is designed to be horizontally moveable on the bearing 17 so that by retracting the rod 16 toward the center of the treatment chamber 2, the container 22 enters the area of the treatment chamber 2. Then it can be moved upward by displacement of the bearing 17 along the rod 23 so that the electrode 24 and/or the microwave conductor 24 and/or the tube 24 protrudes into the interior of the container 22. Here and below, a microwave conductor is considered as being representative of any other coating devices. In this raised position, the container 22 is in its treatment position. The holder of the container 22 in the treatment chamber 2 is attached on the whole to the hollow body 25. The hollow body 25 is supported so that it can rotate (see bearing 29) so that the grippers with the containers 22 can revolve together with the hollow body 25 in the treatment chamber 2.

A container 22 in the treatment position is shown at the left of FIG. 5. The electrode and/or the microwave conductor 24 is arranged in the interior of the container 22. The bearing 17 with the rod 16 and the grippers 15 is shown in a raised position.

The hollow body 25 is connected to the environment via accesses 26, 31. Supply lines such as a voltage supply line for a microwave generator 27 or a gas supply 28 can be passed through the access 26. Supply lines for cooling fluids, for example, can be passed through the access 31. Access is accomplished by means of rotary guides that are impermeable to magnetic fluid. The hollow body 25 can be drive via drive shaft 31. Below the hollow body 25 (e.g., in the case of bearings 29) and above the hollow body 25 a vacuum may be provided, bordered by the outside wall of the treatment chamber 2, as shown here.

A gas inlet 28 as illustrated in FIG. 5 may, however, also be arranged on the stationary part of the treatment chamber 2 so that the introduction of the process gas is facilitated.

It is advantageous in particular if several inlets for different fluids are available to thereby attain a suitable fluid mixture.

Microwave generators or high-frequency generators may be used for generating the plasma. Corresponding electrodes, waveguides, magnets or the like are indicated only schematically or not at all in the figures for the sake of simplicity.

An optional bottom 30 is drawn at the left in FIG. 5. Below the bottom 30 is the interior, which is needed on the right side for input and discharge of the containers 22 through airlocks. However, on the left side (where the treatment of the containers 22 takes place), this lower space is not necessary, so it can be sealed by a bottom 30. Then the volume beneath the bottom 30 may also be omitted.

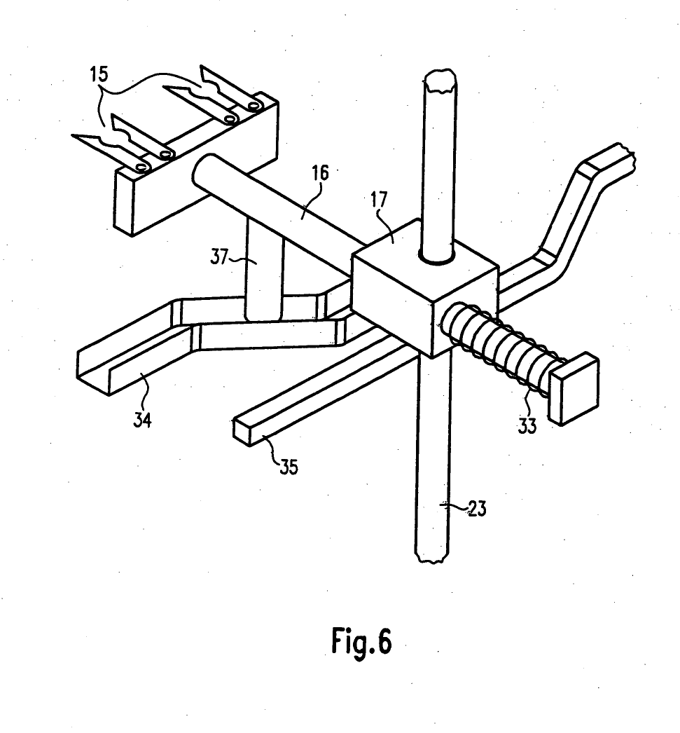

The double grippers are shown in detail in FIG. 6. A bearing 17 is arranged on a rod 23 so that it is adjustable in height. A rod 16 is provided in a horizontally movable arrangement in the bearing 17. At the end of the rod 16 two grippers 15 are provided over a plate so that the mechanism consisting of the rod 16, the bearing 17 and the rod 23, each supported in a non-twisting manner, is required only once for two grippers. The two rods 16 and 23 are provided with a slip coating of tungsten disulfide, which allows maintenance-free use in the treatment chamber. Due to this type of double grippers, the mechanical applications are reduced in comparison with single grippers. For the sake of simplicity, FIG. 6 does not show any mechanism or operating means for opening and closing the grippers such as radial cams. Furthermore, the grippers 15 are shown in a simplified diagram.

For horizontal adjustment of the grippers 15, a cam path 34 is provided with a guide member 37 of the rod 16 protruding into it. FIG. 6 shows the radial cam 34 in such a way that it can displace the rod 16 toward the right as well as toward the left. However, it is also possible for the rod 16 to be prestressed into one of the two directions by a spring 33 so that the radial cam 34 need only be designed to induce the movement of the rod against the spring force of the spring 33. A second radial cam 35 is provided for adjusting the vertical position of the bearing 17 on the rod 23. In doing so the bearing 17 is secured in its height position by contact with the radial cam 35. A force for a movement downward is created either by gravity or by additional spring or by an additional radial cam.

In principle it is also possible to provide a single radial cam which leads the rod 16 in the horizontal direction and the bearing 17 in the vertical direction. For example, if the radial cam 34 were pulled upward, as shown in FIG. 6 for the radial cam 35 at the right rear, then the rod 16 would be raised together with the bearing 17.

To perform the method, containers 22 are transferred via transfer stars 5 to the grippers 7 of the airlock chamber 3. Since the grippers 7 are not arranged equidistantly along the circumference of the airlock chamber 3, the transfer star 5 must be designed accordingly to be able to transfer the containers 22 to the grippers 7 of the airlock chamber 3 with a large and a small intermediate spacing in alternation. The bottles arranged in a cell 8 of the airlock chamber 3 rotate in the clockwise direction 12 from the feed area 10 to the discharge location 11 while they are being evacuated. To do so, the interior of the cell 8 is evacuated by means of the pumps 13. The pressure in the cells 8 is less than 1 millibar, e.g., approximately 0.1 millibar, in the area of the discharge location 11.

In the area of the discharge location 11 (corresponding to reference numeral 36 in FIG. 3), the double grippers of the treatment chamber 2 (see reference numeral 19) move outward and transfer the containers 22 from a cell 8 of the airlock chamber 3. Then the double grippers move back in the direction of the center of the treatment chamber 2 and then move upward so that containers 22 are guided over the electrode 24 and/or the microwave conductor 24 and/or the tube 24. Then the containers 22 are guided clockwise through the treatment chamber 2, whereupon they pass through the plasma areas. In these plasma areas, the inside and/or outside of the containers 22 is/are sterilized and/or provided with a coating. The coating of the container 22 serves to reduce the gas permeability for CO₂, for example, to thereby lengthen the minimum shelf life of carbonated beverages.

A low-pressure plasma (approx. 0.1 Pa) may be used as the plasma because it is “cold” and therefore does not damage PET containers, for example. Suitable process gases include, for example, argon, oxygen, carbon dioxide, hydrogen, nitrogen, ammonia or air.

For coating the containers 22 on the inside, a reactive material is provided in the interior of the containers 22. This may be accomplished with the electrodes 24 or tubes 24. Quartz glass rods 24 result in a coating of SiO₂ on the inside of containers 22 which is visually transparent and safe for foods.

It is also advantageous to apply TiO₂ which is advantageously bound as nanoparticles in an SiO₂ matrix.

TiO₂ is photochemically active. This means that with exposure to UV radiation, preferably from the outside of the container (with a wavelength between 200 and 400 nm, for example), TiO₂ has a high oxidation potential and is capable of oxidizing organic molecules (e.g., microbes).

Through a suitable choice of the process gases and/or suitable coating material, it is thus possible to provide for both coating and sterilization of containers on the inside and/or outside simultaneously or in succession in the treatment chamber 2.

After or during application of an SiO₂ layer, preferably TiO₂ in the form of nanoparticles (where the TiO₂ is preferably in the anatase crystal modification), sterilization may be performed by exposure to UV light. The UV light may originate from the plasma itself or may be generated by UV lamps. The UV lamps may also be arranged here in the area of the output airlock, for example, to thereby be able to utilize the discharge time for sterilization as well. The UV lamps may also illuminate the containers 22 in the area where the bottles are lowered (see reference numeral 21 in FIG. 4).

It is also possible for a plasma to be ignited in a first half of the treatment chamber 4, for example, causing the containers 22 to be coated, and then a plasma is ignited in the second half, performing the sterilization of the containers. The reverse procedure is also possible.

However, it is advantageous if the coating and the sterilization take place simultaneously because then enough time is available for both processes. One fact to be taken into account here is that with a diameter of the treatment chamber 2 of approximately two to three meters and a desired bottle throughput of 20,000 to 30,000 bottles per hour, the dwell time of the containers 22 in the treatment chamber 2 is only a few seconds, i.e., on the order of less than 10 seconds, e.g., 5 seconds. Adequate sterilization and coating of the containers must be ensured within this very short period of time.

After discharge of the containers 22 out of the airlock 4, they may advantageously be conveyed further over a sterile conveyor, e.g., to a filler.

The various mechanical design aspects, e.g., the height of the airlocks 3, 4 in comparison with the treatment chamber 2, the cells 8 for at least two containers 22, the hollow body 25 in vacuo, the changes in height of the containers to bring them into treatment position, the plasma treatment area 32 which is in the shape of a ring segment, the various bottom heights in the plasma treatment area 32, the various radial cams 34, 35 for the grippers 15, etc. are advantageously independent of whether the method and/or the treatment chamber 2 are provided for coating and sterilization or for only coating or sterilization.

We claim:

1. Device for treating containers with a plasma, comprising a treating device (1) that is designed for at least one of sterilizing or coating the containers (22).

2. Device according to claim 1, characterized in that the treating device (1) comprises a treatment chamber (2).

3. Device according to claim 2, and a rotor (25) is provided in the treatment chamber (2), serving to transport the containers during the treatment.

4. Device according to claim 3, wherein the rotor (25) has grippers (15) for containers (22).

5. Device according to claim 41, wherein the double grippers (15) are arranged in such a way that the respective container positions have the same angular distance from neighboring container positions.

6. Device according to claim 4, wherein the grippers (15) are adjustable in height.

7. Device according to claim 6, wherein one of electrodes (24), microwave conductors (24), coating materials (24), and a combination thereof are provided for generating the plasma.

8. Device according to claim 4, wherein the grippers (15) are adjustable horizontally.

9. Device according to claim 3, wherein the rotor (25) comprises a central co-rotating hollow body (25) whose diameter preferably amounts to at least one of approximately 5%, 10%, 20%, 30%, 40%, 50%, 75%, 80%, 85%, and 90% of the diameter of the treatment chamber (2).

10. Device according to claim 9, wherein the hollow body (25) has a continuous connection to the atmosphere.

11. Device according to claim 9, wherein supply lines (28) for the interior of the treatment chamber (2) are passed through the wall of the hollow body (25).

12. Device according to claim 1, wherein one of a ring-shaped and ring-segment-shaped plasma treatment area (32) is provided.

13. Device according to claim 12, wherein the plasma treatment area (32) is bordered at the outside by a stationary device wall within which there is a co-rotating hollow body (25).

14. Device according to claim 12, wherein the bottom (30) of the plasma treatment area (32) has at least two different height levels.

15. Device according to claim 14, wherein the area of the beginning and the end of the plasma treatment area (32) the bottom is lower than another part of the plasma treatment area (32).

16. Device according to claim 2, and wherein one of one and two airlock chambers (3, 4) are provided for one of input and discharge into or from the treatment chamber (2).

17. Device according to claim 16, wherein each of the one or two airlock chambers (3, 4) has cells (8) to receive the containers (22), whereby each cell (8) serves to receive at least two containers (22).

18. Device according to wherein each cell (8) has a gripper (7) for gripping the containers (22) of a cell (8).

19. Device according to claim 16, wherein two different levels are provided for conveyance of the containers (22) in the one or two airlocks (3, 4) and in the treatment chamber (2).

20. Device according to claim 16, and transfer stars (5, 6) are provided at the airlock chambers (3, 4) with which containers (22) can be transported one of toward the airlock chambers (3) and away from the airlock chambers (4).

21. Device according to claim 16, and wherein UV lamps are provided with which the containers can be exposed to UV light in the area of the airlock (4) with which the containers (22) can be discharged from the treatment chamber (2).

22. Device according to claim 1, and wherein devices for generating at least two different plasmas are provided.

23. Device according to claim 22, and wherein at least two gas inlets are provided for two different gases.

24. Device according to claim 22, and wherein two different sections are provided for generating two different plasmas.

25. Device according to claim 2, and wherein UV lamps are provided for illuminating the containers (22) in one of the area of the treatment chamber (2) and in the area of the transfer of the containers (22) out of the treatment chamber (2).

26. Device according to claim 1, and wherein one and the same plasma may be used for both coating and sterilization.

27. Method for treating containers with a plasma, comprising at least one of sterilizing the containers (22), coating of the containers (22), and the combination of sterilizing and coating of the containers (22).

28. Method according to claim 27, wherein the sterilizing and coating are performed in the same device (1).

29. Method according to claim 27, wherein the sterilizing and the coating are performed simultaneously.

30. Method according to claim 27, wherein the process gases used are one of Ar, O₂, CO₂, H₂, N₂, NH₃, air, or a mixture thereof.

31. Method according to claim 27, wherein the coating comprises one of SiO₂, TiO₂ or a mixture thereof.

32. Method according to claim 27, wherein the sterilization comprises exposure to UV light.

33. Method according to claim 32, wherein the UV light is generated by a plasma.

34. Method according to claim 32 wherein the UV light is generated by UV lamps for the UV light exposure.

35. Airlock for containers, comprising an airlock having cells to receive the containers, and wherein at least one cell (8) is formed to receive at least two containers (22).

36. Airlock according to claim 35, wherein one of vacuum pumps (13) and connections for vacuum pumps are provided along the circumference of the airlock.

37. Airlock according to claim 35, and wherein gaskets (9) are provided and which seal the cells from the outside.

38. Airlock according to claim 35, and wherein gaskets (9) are provided and which seal the cells from the airlock bottom and airlock cover.

39. Device according to claim 1, wherein the containers are PET bottles.

40. Device according to claim 2, wherein the treatment chamber (2) is a low pressure plasma chamber.

41. Device according to claim 4, wherein the grippers (15) are designed as double grippers for simultaneously gripping two containers (22).

42. Device according to claim 6, wherein a radial cam (35) is provided for the height adjustment.

43. Device according to claim 7, wherein the height adjustment is performed in such a way that the containers (22) can be moved between a first position and a second position whereby in the first position the one of the electrodes, microwave conductors (24), the coating material (24), and the combination thereof is outside of the container and is at least partially inside the container (22) when in the second position.

44. Device according to claim 8, wherein a radial cam (34) is provided for the horizontal adjustment.

45. Device according to claim 8, wherein a radial cam (34) is provided for the horizontal adjustment and preferably the horizontal adjustment is provided for one of receiving, discharging, or a combination thereof of the containers (22).

46. Device according to claim 11, wherein the supply lines (28) are one of gas feed lines, gas discharge lines, cooling water feed lines, cooling water discharge lines, high voltage supply lines, and high-frequency supply lines.

47. Device according to claim 13, wherein the co-rotating hollow body (25) is provided in its interior for atmospheric pressure to prevail and through which the supply lines (28) can pass.

48. Device according to claim 16, wherein the airlock chambers (3, 4) have grippers (7) for containers (22) which are arranged on a rotatable rotor.

49. Device according to claim 17, wherein each cell (8) receives exactly two containers (22).

50. Method according to claim 27, wherein the containers are PET bottles.

51. Method according to claim 31, wherein the TiO₂ is in the anatase crystal modification, whereby the TiO₂ is incorporated as nanoparticles into an SiO₂ matrix.

52. Method according to claim 33, wherein the plasma is the plasma used for coating.

53. Airlock according to claim 35, wherein each cell (8) is formed to receive exactly two containers.

54. Airlock according to claim 35, wherein the containers are PET bottles.
